SEQUENCE LISTING (21 sequences)

SEQ ID NO: 1 - RNA, 22 nt, artificial sequence
  synthetic RNA
ccacaugaagcagcacgacuuc

SEQ ID NO: 2 - RNA, 22 nt, artificial sequence
  synthetic RNA
gaagucgugcugcuucaugugg

SEQ ID NO: 3 - RNA, 21 nt, artificial sequence
  synthetic RNA
ccacaugaagcagcacgacuu

SEQ ID NO: 4 - RNA, 21 nt, artificial sequence
  synthetic RNA
gucgugcugcuucaugugguc

SEQ ID NO: 5 - RNA, 22 nt, artificial sequence
  synthetic RNA
acaggaugaggaucguuucgca

SEQ ID NO: 6 - RNA, 22 nt, artificial sequence
  synthetic RNA
ugcgaaacgauccucauccugu

SEQ ID NO: 7 - RNA, 21 nt, artificial sequence
  synthetic RNA
gaugaggaucguuucgcauga

SEQ ID NO: 8 - RNA, 21 nt, artificial sequence
  synthetic RNA
augcgaaacgauccucauccu

SEQ ID NO: 9 - RNA, 21 nt, artificial sequence
  synthetic RNA
gaugaggaucguuucgcauga

SEQ ID NO: 10 - RNA, 23 nt, artificial sequence
  synthetic RNA
ucaugcgaaacgauccucauccu

SEQ ID NO: 11 - RNA, 21 nt, artificial sequence
  synthetic RNA
ccacaugaagcagcacgacuu

SEQ ID NO: 12 - RNA, 23 nt, artificial sequence
  synthetic RNA
aagucgugcugcuucaugugguc

SEQ ID NO: 13 - RNA, 21 nt, artificial sequence
  siRNA
aagcagaguucaaaagcccuu

SEQ ID NO: 14 - RNA, 23 nt, artificial sequence
  siRNA
aagggcuuuugaacucugcuuaa

SEQ ID NO: 15 - RNA, 40 nt, artificial sequence
  fusion sequence of BCR-ABL mRNA
uggauuuaagcagaguucaaaagcccuucagcggccagau

SEQ ID NO: 16 - DNA, 20 bp, artificial sequence
  PRIMER
agcacggacagactcatggg

SEQ ID NO: 17 - DNA, 20 bp, artificial sequence
  PRIMER
gctgccagtctctgtcctgc

SEQ ID NO: 18 - DNA, 22 bp, artificial sequence
  PRIMER
agggccaggtccagctggaccc

SEQ ID NO: 19 - DNA, 19 bp, artificial sequence
  PRIMER
ggctgtcctcgtcctccag

SEQ ID NO: 20 - DNA, 21 bp, artificial sequence
  PRIMER
tcagaccctgaggctcaaagt

SEQ ID NO: 21 - DNA, 24 bp, artificial sequence
  PRIMER
atctggaagaagcccttcagcggc

(19) United States
(12) Patent Application Publication
(10) Pub. No.: US 2004/0038921 A1
(43) Pub. Date: Feb. 26, 2004

(54) Composition and method for inhibiting expression of a target gene

(75) Inventors: Roland Kreutzer, Weidenberg (DE); Stefan Limmer, Neudrossenfeld (DE); Sylvia Limmer, Neudrossenfeld (DE); Philipp Hadwiger, Bayreuth (DE)

Correspondence Address: PALMER & DODGE, LLP, KATHLEEN M. WILLIAMS, 111 HUNTINGTON AVENUE, BOSTON, MA, 02199, US

(73) Assignee: Ribopharma AG

(21) Appl. No.: 10/382,634

(22) Filed: Aug. 11, 2003

Related U.S. Application Data
(63) Continuation-in-part of application No. PCT/EP02/11971, filed on Oct. 25, 2002.

(30) Foreign Application Priority Data
Oct. 26, 2001 (DE) 101 55 280.7
Nov. 29, 2001 (DE) 101 58 411.3
Dec. 7, 2001 (DE) 101 60 151.4
Jul. 9, 2002 (DE) 102 30 996.5
Jan. 9, 2002 (WO) PCT/EP02/00152
Jan. 9, 2002 (WO) PCT/EP02/00151
Oct. 25, 2002 (WO) PCT/EP02/11971

(51) Int. Cl. A61K 48/00

(52) U.S. Cl. 514/44

(57) ABSTRACT

The present invention relates to pharmaceutical compositions comprising a double-stranded oligoribonucleic acid (dsRNA) having a nucleotide sequence which is substantially identical to at least a part of a target gene in a mammalian cell and which is less than 25 nucleotides in length, together with a pharmaceutically acceptable carrier. The pharmaceutical compositions are useful for inhibiting the expression of a target gene, as well as for treating diseases caused by expression of the target gene, in a mammal at very low dosages (i.e., less than 5 milligrams, preferably less than 25 micrograms, per kg body weight per day). The invention also relates to methods for inhibiting the expression of a target gene in a mammal, as well as methods for treating diseases caused by expression of the gene.

### RELATED APPLICATIONS

This application is a continuation-in-part of International Application No. PCT/EP02/11971, which designated the United States and was filed on Oct. 25, 2002, which claims the benefit of German Patent No. 101 55 280.7, filed on Oct. 26, 2001, German Patent No. 101 58 411.3, filed on Nov. 29, 2001, German Patent No. 101 60 151.4, filed on Dec. 7, 2001, EP Patent No. PCT/EP02/00152, filed on Jan. 9, 2002, EP Patent No. PCT/EP02/00151, filed on Jan. 9, 2002, and German Patent No. 102 30 996.5, filed on Jul. 9, 2002. The entire teachings of the above application(s) are incorporated herein by reference.

### BACKGROUND OF THE INVENTION

A number of therapeutic agents which inhibit expression of a target gene are known in the art, including antisense RNA (Skorski, T. et al., Proc. Natl. Acad. Sci. USA (1994) 91:4504-4508) and hammerhead-based ribozymes (James, H. A, and I. Gibson, Blood (1998) 91:371). However, both of these agents have inherent limitations. Antisense approaches, using either single-stranded RNA or DNA, act in a 1:1 stoichiometric relationship and thus have low efficacy, as well as questionable specificity (Skorski et al., supra). For example, Jansen, B., et al., The Lancet (2000) 356:1728-1733, discloses the administration of antisense nucleotides to patients in dosages of from 0.6 to 6.5 mg/kg per day. Long-term plasma concentrations of proteins above 1 mg/L are considered biologically significant. Jansen et al. reports that while a dosage of 0.6 mg/kg per day had no effect on the concentration of proteins encoded by the target gene, a plasma concentration of 1 mg/L protein is possible using a dose of 2 mg/kg body weight per day of antisense oligoribonucleotides. However, the treatment is successful in only a fraction of patients.

Hammerhead ribozymes, which because of their catalytic activity can degrade a higher number of target molecules, have been used to overcome the stoichiometry problem associated with antisense RNA. Thus, at least theoretically, the use of hammerhead ribozymes should reduce the dosage required to achieve inhibition of expression of the target gene. However, hammerhead ribozymes require specific nucleotide sequences in the target gene, which are not always present.

More recently, double-stranded RNA molecules (dsRNA) have been shown to block gene expression in a highly conserved regulatory mechanism known as RNA interference (RNAi). Briefly, the RNAse III Dicer processes dsRNA into small interfering RNAs (siRNA) of approximately 22 nucleotides, which serve as guide sequences to induce target-specific mRNA cleavage by an RNA-induced silencing complex RISC (Hammond, S. M., et al., Nature (2000) 404:293-296). In other words, RNAi involves a catalytic-type reaction whereby new siRNAs are generated through successive cleavage of long dsRNA. Thus, unlike antisense, RNAi degrades target RNA in a non-stoichiometric manner. When administered to a cell or organism, exogenous dsRNA has been shown to direct the sequence-specific degradation of endogenous messenger RNA (mRNA) through RNAi. WO 99/32619 (Fires et al.) discloses the use of a dsRNA of at least 25 nucleotides in length to inhibit the expression of a target gene in C. elegans. Sharp, P. A., Genes & Dev. (2001) 15:485-490, suggests that dsRNA from a related but not identical gene (i.e., >90% homologous) can be used for gene silencing if the dsRNA and target gene share segments of identical and uninterrupted sequences of significant length, i.e., more than 30-35 nucleotides. Unfortunately, the use of long dsRNAs in mammalian cells to elicit RNAi is usually not practical, due to the deleterious effects of the interferon response, as well as the problems associated with the intracellular delivery of large molecules.

Thus, despite significant advances in the field, there remains a need for a therapeutic agent that can effectively inhibit expression of a target gene at a reasonably low dose. In particular, agents that are small enough for efficient intracellular delivery, and which have both high efficacy (hence are effective at low dosages) and high specificity for the target gene would be therapeutically beneficial. Pharmaceutical compositions comprising such agents would be useful for treating diseases caused by the expression of a target gene.

### SUMMARY OF THE INVENTION

The present invention discloses a pharmaceutical composition for inhibiting the expression of a target gene in a mammal, as well as treating diseases caused by expression of the gene. The composition comprises a double-stranded oligoribonucleic acid (dsRNA) comprising a nucleotide sequence of less than 25 nucleotides which is substantially identical to at least a part of a target gene in a mammalian cell, together with a pharmaceutically acceptable carrier. The present invention also discloses a method for inhibiting the expression of a target gene in a mammal, and a method of treatment, using the above-described pharmaceutical composition. The pharmaceutical compositions and methods of the present invention are useful for treating diseases caused by the expression of a target gene.

In one aspect, the invention relates to a pharmaceutical composition for inhibiting the expression of a target gene in a mammal. The composition comprises a double-stranded ribonucleic acid (dsRNA) and a pharmaceutically acceptable carrier, wherein the dsRNA comprises a nucleotide sequence which is substantially identical to at least a part of the target gene and which is less than 25 nucleotides in length, and wherein the pharmaceutical composition is in a unit dosage amount of less than 5 milligram (mg) of dsRNA per kg body weight of the mammal.

The pharmaceutical composition may have a dosage unit amount of dsRNA of 0.01 to 2.5 milligrams (mg), 0.1 to 200 micrograms (μg), 0.1 to 100 μg, 1.0 to 50 μg, or 1.0 to 25 μg per kilogram body weight. Preferably, dosage unit amount of dsRNA is less than 25 μg per kilogram body weight.

The dsRNA of the pharmaceutical composition of the invention may comprise a complementary RNA strand having a complementary nucleotide sequence which is complementary to an mRNA transcript of a portion of the target gene. The complementary nucleotide sequence may be 19 to 24 nucleotides in length, 20 to 24 nucleotides in length, or 21 to 23 nucleotides in length. Preferably, the complementary nucleotide sequence is 22 or 23 nucleotides in length. The complementary RNA strand may be 1 to 30 nucleotides in length, 21 to 25 nucleotides in length, or 21 to 24 nucleotides in length. Preferably, the complementary RNA strand is 23 nucleotides in length. The dsRNA may comprise a first complementary RNA strand and a second RNA strand, wherein the first complementary RNA strand comprises a complementary nucleotide sequence which is complementary to an RNA transcript of a portion of the target gene, and wherein each of the first and second RNA strands comprise a 3′-terminus and a 5′-terminus. At least one of the RNA strands may comprise a nucleotide overhang of 1 to 4 nucleotides in length. Preferably, the nucleotide overhang is one or two nucleotides in length and is on the 3′-terminus of the first complementary RNA strand. The dsRNA further may comprise first and second ends. The first end may comprise the 3′-terminus of the first complementary RNA strand and the 5′-terminus of the second RNA strand, and the second end may comprise the 5′-terminus of the first complementary RNA strand and the 3′-terminus of the second RNA strand. The first end may comprise a nucleotide overhang on the 3′-terminus of the first complementary RNA strand, and the second end may be blunt. The first complementary RNA strand may be 23 nucleotides in length and comprise a 2-nucleotide overhang at the 3′-terminus, and the second RNA strand may be 21 nucleotides in length, and the second end of the dsRNA may be blunt.

The pharmaceutically acceptable carrier of the pharmaceutical composition may be an aqueous solution, such as a phosphate buffered saline. In another embodiment, the pharmaceutically acceptable carrier may comprise a micellar structure, such as a liposome, capsid, capsoid, polymeric nanocapsule, or polymeric microcapsule. The polymeric nanocapsule or microcapsule may comprise polybutylcyanoacrylate. The pharmaceutical composition may be formulated to be administered by inhalation, infusion, injection, or orally, preferably by intravenous or intraperitoneal infusion or injection.

In another aspect, the invention relates to a method for inhibiting the expression of a target gene in a mammal, by administering a pharmaceutical composition according to the invention. The gene to be inhibited may be, for example, an oncogene; cytokinin gene; idiotype protein gene (Id protein gene); prion gene; gene that expresses molecules that induce angiogenesis, adhesion molecules, and cell surface receptors; genes of proteins that are involved in metastasizing and/or invasive processes; genes of proteases as well as of molecules that regulate apoptosis and the cell cycle; genes that express the EGF receptor; the multi-drug resistance 1 gene (MDR1 gene); a gene or component of a virus, particularly a human pathogenic virus, that is expressed in pathogenic organisms, preferably in plasmodia.

In still another aspect, the invention relates to a method for treating a disease caused by the expression of a target gene in a mammal, by administering a pharmaceutical composition as described above. The gene to be inhibited may be any gene which causes disease, including those described elsewhere herein. The disease to be treated may be any disease caused by the expression of a target gene. For example, the disease may be a cellular proliferative and/or differentiative disorders such as a cancer (e.g., carcinoma, carcoma, metastatic disorders or hematopoietic neoplastic disorders, such as leukemias); an immune disorder, such as those associated with overexpression of a gene or expression of a mutant gene (e.g., autoimmune diseases, such as diabetes mellitus, arthritis (including rheumatoid arthritis, juvenile rheumatoid arthritis, osteoarthritis, psoriatic arthritis), multiple sclerosis, encephalomyelitis, myasthenia gravis, systemic lupus erythematosis, automimmune thyroiditis, dermatitis (including atopic dermatitis and eczematous dermatitis), psoriasis, Sjogren's Syndrome, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing, loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves' disease, sarcoidosis, primary biliary cirrhosis, uveitis posterior, and interstitial lung fibrosis), graft-versus-host disease, cases of transplantation, and allergy.

### BRIEF DESCRIPTION OF THE FIGURES

FIG. 1 is a GFP-specific immunoperoxidase staining of paraffin kidney sections of transgenic GFP mice.

FIG. 2 is a GFP-specific immunoperoxidase staining of paraffin heart sections of transgenic GFP mice.

FIG. 3 is a GFP-specific immunoperoxidase staining of paraffin pancreas sections of transgenic GFP mice.

FIG. 4 is a Western blot analysis of GFP expression in plasma.

FIG. 5 is a Western blot analysis of GFP expression in kidney.

FIG. 6 is a Western blot analysis of GFP expression in heart.

FIG. 7 is the percent of GFP expression (FACS analysis) in the blood of GFP transgenic mice after treatment with specific (GFP group) and non-specific (control group) dsRNA.

FIG. 8 shows the GFP expression level (FACS analysis) in the blood of individual animals after treatment with specific (GFP group) and non-specific (control group) dsRNA.

FIG. 9 shows a FACS analysis of expressed surface marker proteins CD11b, CD3, CD4, CD8a, and CD19.

FIG. 10 is a Western blot analysis of GFP expression in the blood of animals 4-7 treated with specific dsRNA (GFP group, 4 tracks left), and of animals 3-6 treated with non-specific dsRNA (control group, 4 tracks right).

### DETAILED DESCRIPTION OF THE INVENTION

The present invention relates to pharmaceutical compositions and methods for treating diseases caused by the expression of a target gene, as well as method for inhibiting the expression of a target gene in a mammal using a double-stranded RNA (dsRNA). dsRNA directs the sequence-specific degradation of mRNA through a process known as RNA interference (RNAi). The process occurs in a wide variety of organisms, including mammals and other vertebrates. Using transgenic mice, the present inventors have demonstrated that very low dosages of short dsRNA can specifically and efficiently mediate RNAi, resulting in significant inhibition of expression of the target gene (transgene). The present invention encompasses compositions comprising these short dsRNAs and their use for specifically inactivating gene function. The use of these dsRNAs enables the degradation of mRNAs of target genes which are implicated in disease processes. Thus, the methods and compositions of the present invention comprising these dsRNAs are useful for treating diseases caused by the expression of the target gene.

The following detailed description discloses how to make and use pharmaceutical compositions comprising dsRNA to inhibit the expression of a target gene, as well as pharmaceutical compositions for treating diseases caused by the expression of a target gene. The pharmaceutical compositions of the present invention comprise a dsRNA having a nucleotide sequence of less than 25 nucleotides, which is substantially identical to at least a part of the target gene, together with a pharmaceutically acceptable carrier. The dsRNA preferably has a single-stranded nucleotide overhang of two or three nucleotides at the 3′-terminal end of the RNA strand that is complementary to an mRNA transcript of the target gene.

Accordingly, certain aspects of the present invention relate to pharmaceutical compositions comprising the dsRNA of the present invention together with a pharmaceutically acceptable carrier, methods of using the compositions to inhibit expression of a target gene, and methods of using the pharmaceutical compositions to treat diseases caused by a target gene.

I. Definitions

For convenience, the meaning of certain terms and phrases used in the specification, examples, and appended claims, are provided below.

As used herein and as known in the art, the term “identity” is the relationship between two or more polynucleotide sequences, as determined by comparing the sequences. Identity also means the degree of sequence relatedness between polynucleotide sequences, as determined by the match between strings of such sequences. Identity can be readily calculated (see, e.g, Computation Molecular Biology, Lesk, A. M., eds., Oxford University Press, New York (1998), and Biocomputing: Informatics and Genome Projects, Smith, D. W., ed., Academic Press, New York (1993), both of which are incorporated by reference herein). While there exist a number of methods to measure identity between two polynucleotide sequences, the term is well known to skilled artisans (see, e.g., Sequence Analysis in Molecular Biology, von Heinje, G., Academic Press (1987); and Sequence Analysis Primer, Gribskov., M. and Devereux, J., eds., M Stockton Press, New York (1991)). Methods commonly employed to determine identity between sequences include, for example, those disclosed in Carillo, H., and Lipman, D., SIAM J. Applied Math. (1988) 48:1073. “Substantially identical,” as used herein, means there is a very high degree of homology (preferably 100% sequence identity) between the inhibitory dsRNA and the corresponding part of the target gene. However, dsRNA having greater than 90%, or 95% sequence identity may be used in the present invention, and thus sequence variations that might be expected due to genetic mutation, strain polymorphism, or evolutionary divergence can be tolerated. Although 100% identity is preferred, the dsRNA may contain single or multiple base-pair random mismatches between the RNA and the target gene.

As used herein, “target gene” refers to a section of a DNA strand of a double-stranded DNA that is complementary to a section of a DNA strand, including all transcribed regions, that serves as a matrix for transcription. The target gene is therefore usually the sense strand. A target gene may also be a part of a viral genome, including the genome of a (+) strand RNA virus, such as a hepatitis C virus.

The term “complementary RNA strand” refers to the strand of the dsRNA which is complementary to an mRNA transcript that is formed during expression of the target gene, or its processing products. The complementary RNA strand is preferably less than 30, more preferably less than 25, even more preferably 21 to 24, and most preferably 23 nucleotides in length. “dsRNA” refers to a ribonucleic acid molecule having a duplex structure comprising two complementary and anti-parallel nucleic acid strands. Not all nucleotides of a dsRNA must exhibit Watson-Crick base pairs. The maximum number of base pairs is the number of nucleotides in the shortest strand of the dsRNA.

As used herein, the term “complementary nucleotide sequence” refers to the region on the complementary RNA strand which is complementary to an mRNA transcript of a portion of the target gene. The complementary nucleotide sequence comprises less than 25 nucleotides, preferably 19 to 24 nucleotides, more preferably 20 to 24, even more preferably 21 to 23, and most preferably 22 or 23 nucleotides. dsRNAs of this length are particularly efficient in inhibiting the expression of the target gene. “Introducing into” means uptake or absorption in the cell, as is understood by those skilled in the art. Absorption or uptake can occur through cellular processes, or by auxiliary agents or devices.

As used herein, a “pharmaceutical composition” comprises a pharmacologically effective amount of a dsRNA and a pharmaceutically acceptable carrier. As used herein, “pharmacologically effective amount,” “therapeutically effective amount” or simply “effective amount” refers to that amount of an RNA effective to produce the intended pharmacological, therapeutic or preventive result. For example, if a given clinical treatment is considered effective when there is at least a 25% reduction in a measurable parameter associated with a disease or disorder, a therapeutically effective amount of a drug for the treatment of that disease or disorder is the amount necessary to effect at least a 25% reduction in that parameter.

The term “pharmaceutically acceptable carrier” refers to a carrier for administration of a therapeutic agent. Such carriers include, but are not limited to, saline, buffered saline, dextrose, water, glycerol, ethanol, and combinations thereof. The term specifically excludes cell culture medium. For drugs administered orally, pharmaceutically acceptable carriers include, but are not limited to pharmaceutically acceptable excipients such as inert diluents, disintegrating agents, binding agents, lubricating agents, sweetening agents, flavouring agents, colouring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract.

II. Pharmaceutical Compositions Comprising dsRNA

In one embodiment, the invention relates to a pharmaceutical composition comprising an RNA having a double-stranded structure and a nucleotide sequence which is substantially identical to at least a part of the target gene. The dsRNA comprises two complementary RNA strands, one of which comprises a nucleotide sequence which is substantially identical to a portion of the target gene. The complementary region of the dsRNA comprises less than 25 nucleotides in length. Preferably, the complementary region of the dsRNA is 19 to 24 nucleotides, more preferably 20 to 24, even more preferably 21 to 23, and most preferably 22 or 23 nucleotides in length.

The dsRNA in the composition of the invention may consist of only one strand (the “S1” strand), in which case one end of the dsRNA comprises the 3′- and 5′-termini of the strand and the other end forms a loop structure. dsRNA that consists of two separate strands (i.e., the complementary RNA (S1) strand and a second RNA strand (referred to herein as the “S2” strand), which is complementary to the S1 strand) have two ends. As used herein, an “end” of a dsRNA refers to the tail or terminus of the duplex structure, i.e., where the 5′-end of one RNA strand meets the 3′-end of the other RNA strand in a two stranded structure or, in the case of a single S1 strand, where the 5′- and 3′-ends meet.

In a preferred embodiment, at least one end of the dsRNA has a single-stranded nucleotide overhang of 1 to 4, preferably 2 or 3 nucleotides. As used herein, a “nucleotide overhang” refers to the unpaired nucleotide or nucleotides that protrude from the duplex structure when the 3′-terminal end of one RNA strand extends beyond the 5′-terminus end of the other strand, or vice versa. dsRNAs having at least one nucleotide overhang have unexpectedly superior inhibitory properties than their blunt-ended counterparts. Morover, the present inventors have discovered that the presence of only one nucleotide overhang strengthens the interference activity of dsRNA, without effecting the overall stability of the structure. dsRNA having only one overhang has proven particularly stable and effective in vivo, as well as in a variety of cells, cell culture mediums, blood, and serum. Preferably, the single-stranded overhang is located at the 3′-terminal end of the complementary RNA strand (also referred to herein as the “S1” strand). Such a configuration produces a further increase in efficiency of inhibition. Because of this increased efficiency, the dosage of the dsRNA necessary to inhibit expression of the target gene can be reduced to a maximum of 5 milligrams of body weight of the animal or patient per day. That inhibition can be achieved at this level is surprising and unexpected, given the well known mechanisms in mammals, such as humans, that recognize and attack double-stranded nucleic acids as foreign bodies.

The nucleotide sequence on the complementary RNA strand (S1 strand) has less than 25, preferably 19 to 24, more preferably 20 to 24, even more preferably 21 to 23, and most preferably 22 or 23 nucleotides. Such dsRNA are particularly robust gene silencers. The complementary RNA strand of the dsRNA strand preferably has fewer than 30 nucleotides, more preferably fewer than 25 nucleotides, even more preferably 21 to 24 nucleotides, and most preferably 23 nucleotides. Such dsRNA exhibit superior intracellular stability.

In a preferred embodiment, the pharmaceutical composition comprises dsRNA having two individual (S1 and S2) strands. The pharmaceutical composition is particularly effective when (1) the first complementary strand (also referred to as S1 or antisense strand) is 23 nucleotides in length, (2) the second (S2) strand is 21 nucleotides long, and (3) the 3′-end of the complementary (S1) strand has a single-stranded overhang of two nucleotides. In this embodiment, the opposite end of the dsRNA (i.e., at the 5′-terminus of the S1 strand) is blunt. The first complementary (S1) strand can be complementary to the primary or processed RNA transcript of the target gene.

In one embodiment, the invention relates to a pharmaceutical composition for treating a disease caused by expression of a target gene. In this aspect of the invention, the dsRNA of the invention is formulated as described below. The pharmaceutical composition is administered in a dosage sufficient to inhibit expression of the target gene. The present inventors have found that compositions comprising the dsRNA can be administered at an unexpectedly low dosage. Surprisingly, a maximum dosage of 5 mg dsRNA per kilogram body weight per day is sufficient to inhibit or completely suppress expression of the target gene.

In general a suitable dose of dsRNA will be in the range of 0.01 to 2.5 milligrams per kilogram body weight of the recipient per day, preferably in the range of 0.1 to 200 micrograms per kilogram body weight per day, more preferably in the range of 0.1 to 100 micrograms per kilogram body weight per day, even more preferably in the range of 1.0 to 50 micrograms per kilogram body weight per day, and most preferably in the range of 1.0 to 25 micrograms per kilogram body weight per day. Preferably, pharmaceutical composition comprising the dsRNA is administered once daily. However, the therapeutic agent may be dosed as two, three, four, five, six or more sub-doses administered at appropriate intervals throughout the day. In that case, the dsRNA contained in each sub-dose must be correspondingly smaller in order to achieve the total daily dosage. The dosage unit can also be compounded for a single dose over several days, e.g., using a conventional sustained release formulation which provides sustained and consistent release of the dsRNA over a several day period. Sustained release formulations are well known in the art. In this embodiment, the dosage unit contains a corresponding multiple of the daily dose. Regardless of the formulation, the pharmaceutical composition must contain dsRNA in a quantity sufficient to inhibit expression of the target gene in the animal or human being treated. The composition can be compounded in such a way that the sum of the multiple units of dsRNA together contain a sufficient dose.

Toxicity and therapeutic efficacy of dsRNAs can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred.

The data obtained from cell culture assays and animal studies can be used in formulation a range of dosage for use in humans. The dosage of compositions of the invention lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range of the compound or, when appropriate, of the polypeptide product of a target sequence (e.g., achieving a decreased concentration of the polypeptide) that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The pharmaceutical compositions encompassed by the invention may be administered by any means known in the art including, but not limited to oral or parenteral routes, including intravenous, intramuscular, intraperitoneal, subcutaneous, transdermal, airway (aerosol), rectal, vaginal and topical (including buccal and sublingual) administration. In preferred embodiments, the pharmaceutical compositions are administered by intravenous or intraparenteral infusion or injection.

For oral administration, the dsRNAs useful in the invention will generally be provided in the form of tablets or capsules, as a powder or granules, or as an aqueous solution or suspension.

Tablets for oral use may include the active ingredients mixed with pharmaceutically acceptable excipients such as inert diluents, disintegrating agents, binding agents, lubricating agents, sweetening agents, flavouring agents, colouring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract.

Capsules for oral use include hard gelatin capsules in which the active ingredient is mixed with a solid diluent, and soft gelatin capsules wherein the active ingredients is mixed with water or an oil such as peanut oil, liquid paraffin or olive oil.

For intramuscular, intraperitoneal, subcutaneous and intravenous use, the pharmaceutical compositions of the invention will generally be provided in sterile aqueous solutions or suspensions, buffered to an appropriate pH and isotonicity. Suitable aqueous vehicles include Ringer's solution and isotonic sodium chloride. In a preferred embodiment, the carrier consists exclusively of an aqueous buffer. In this context, “exclusively” means no auxiliary agents or encapsulating substances are present which might affect or mediate uptake of dsRNA in the cells that express the target gene. Such substances include, for example, micellar structures, such as liposomes or capsids, as described below. Surprisingly, the present inventors have discovered that compositions containing only naked dsRNA and a physiologically acceptable solvent are taken up by cells, where the dsRNA effectively inhibits expression of the target gene. Although microinjection, lipofection, viruses, viroids, capsids, capsoids, or other auxiliary agents are required to introduce dsRNA into cell cultures, surprisingly these methods and agents are not necessary for uptake of dsRNA in vivo. Aqueous suspensions according to the invention may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

The pharmaceutical compositions useful according to the invention also include encapsulated formulations to protect the dsRNA against rapid elimination from the body. In this embodiment, the dsRNA is surrounded by or bound to a micellar structure, such as a liposome, capsid, capsoid, or polymeric nano- or microcapsule, which facilitate uptake of the dsRNA into the cell. The capsid may be a chemically or enzymatically synthesized capsid, or a natural viral capsid or derivative thereof. The polymeric nano- or microcapsule may comprise at least one biologically degradable polymer, such as polybutylcyanoacrylate. Polymeric nano- or microcapsules facilitate transport and release of the encapsulated or bound dsRNA into the cell. Other biodegradable, biocompatible polymers include, for example, ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811; PCT publication WO 91/06309; and European patent publication EP-A-43075, which are incorporated by reference herein.

In addition to their administration singly, the dsRNAs useful according to the invention can be administered in combination with other known agents effective in treatment of malignant diseases. In any event, the administering physician can adjust the amount and timing of dsRNA administration on the basis of results observed using standard measures of efficacy known in the art or described herein.

For oral administration, the dsRNAs useful in the invention will generally be provided in the form of tablets or capsules, as a powder or granules, or as an aqueous solution or suspension.

III. Methods for Treating Diseases Caused by Expression of a Target Gene.

In one embodiment, the invention relates to a method for treating a subject having a disease or at risk of developing a disease caused by the expression of a target gene. In this embodiment, the dsRNA can act as novel therapeutic agents for controlling one or more of cellular proliferative and/or differentiative disorders, disorders associated with bone metabolism, immune disorders, hematopoietic disorders, cardiovascular disorders, liver disorders, viral diseases, or metabolic disorders. The method comprises administering a pharmaceutical composition of the invention to the patient (e.g., human), such that expression of the target gene is silenced. Because of their high specificity, the dsRNAs of the present invention specifically target mRNAs of target genes of diseased cells and tissues, as described below, and at surprisingly low dosages. The pharmaceutical compositions are formulated as described in the preceding section, which is hereby incorporated by reference herein.

In the prevention of disease, the target gene may be one which is required for initiation or maintenance of the disease, or which has been identified as being associated with a higher risk of contracting the disease. In the treatment of disease, the dsRNA can be brought into contact with the cells or tissue exhibiting the disease. For example, dsRNA substantially identical to all or part of a mutated gene associated with cancer, or one expressed at high levels in tumor cells, e.g. aurora kinase, may be brought into contact with or introduced into a cancerous cell or tumor gene.

Examples of cellular proliferative and/or differentiative disorders include cancer, e.g., carcinoma, sarcoma, metastatic disorders or hematopoietic neoplastic disorders, e.g., leukemias. A metastatic tumor can arise from a multitude of primary tumor types, including but not limited to those of prostate, colon, lung, breast and liver origin. As used herein, the terms “cancer,” “hyperproliferative,” and “neoplastic” refer to cells having the capacity for autonomous growth, i.e., an abnormal state of condition characterized by rapidly proliferating cell growth. These terms are meant to include all types of cancerous growths or oncogenic processes, metastatic tissues or malignantly transformed cells, tissues, or organs, irrespective of histopathologic type or stage of nvasiveness. Proliferative disorders also include hematopoietic neoplastic disorders, including diseases involving hyperplastic/neoplatic cells of hematopoietic origin, e.g., arising from myeloid, lymphoid or erythroid lineages, or precursor cells thereof.

The pharmaceutical compositions of the present invention can also be used to treat a variety of immune disorders, in particular those associated with overexpression of a gene or expression of a mutant gene. Examples of hematopoietic disorders or diseases include, without limitation, autoimmune diseases (including, for example, diabetes mellitus, arthritis (including rheumatoid arthritis, juvenile rheumatoid arthritis, osteoarthritis, psoriatic arthritis), multiple sclerosis, encephalomyelitis, myasthenia gravis, systemic lupus erythematosis, automimmune thyroiditis, dermatitis (including atopic dermatitis and eczematous dermatitis), psoriasis, Sjogren's Syndrome, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing, loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves' disease, sarcoidosis, primary biliary cirrhosis, uveitis posterior, and interstitial lung fibrosis), graft-versus-host disease, cases of transplantation, and allergy.

Examples of genes which can be targeted for treatment include, without limitation, an oncogene (Hanahan, D. and R. A. Weinberg, Cell (2000) 100:57; and Yokota, J., Carcinogenesis (2000) 21(3):497-503); a cytokine gene (Rubinstein, M., et al., Cytokine Growth Factor Rev. (1998) 9(2):175-81); a idiotype (Id) protein gene (Benezra, R., et al., Oncogene (2001) 20(58):8334-41; Norton, J. D., J Cell Sci. (2000) 113(22):3897-905); a prion gene (Prusiner, S. B., et al., Cell (1998) 93(3):337-48; Safar, J., and S. B. Prusiner, Prog. Brain Res. (1998) 117:421-34); a gene that expresses molecules that induce angiogenesis (Gould, V. E. and B. M. Wagner, Hum. Pathol. (2002) 33(11):1061-3); adhesion molecules (Chothia, C. and E. Y. Jones, Annu. Rev. Biochem. (1997) 66:823-62; Parise, L. V., et al., Semin. Cancer Biol. (2000) 10(6):407-14); cell surface receptors (Deller, M. C., and Y. E. Jones, Curr. Opin. Struct. Biol. (2000) 10(2):213-9); genes of proteins that are involved in metastasizing and/or invasive processes (Boyd, D., Cancer Metastasis Rev. (1996) 15(1):77-89; Yokota, J., Carcinogenesis (2000) 21(3):497-503); genes of proteases as well as of molecules that regulate apoptosis and the cell cycle (Matrisian, L. M., Curr. Biol. (1999) 9(20):R776-8; Krepela, E., Neoplasma (2001) 48(5):332-49; Basbaum and Werb, Curr. Opin. Cell Biol. (1996) 8:731-738; Birkedal-Hansen, et al., Crit. Rev. Oral Biol. Med. (1993) 4:197-250; Mignatti and Rifkin, Physiol. Rev. (1993) 73:161-195; Stetler-Stevenson, et al., Annu. Rev. Cell Biol. (1993) 9:541-573; Brinkerhoff, E., and L. M. Matrisan, Nature Reviews (2002) 3:207-214; Strasser, A., et al., Annu. Rev. Biochem. (2000) 69:217-45; Chao, D. T. and S. J. Korsmeyer, Annu. Rev. Immunol. (1998) 16:395-419; Mullauer, L., et al., Mutat. Res. (2001) 488(3):211-31; Fotedar, R., et al., Prog. Cell Cycle Res. (1996) 2:147-63; Reed, J. C., Am. J Pathol. (2000) 157(5):1415-30; D'Ari, R., Bioassays (2001) 23(7):563-5); genes that express the EGF receptor; Mendelsohn, J. and J. Baselga, Oncogene (2000) 19(56):6550-65; Normanno, N., et al., Front. Biosci. (2001) 6:D685-707); and the multi-drug resistance 1 gene, MDR1 gene (Childs, S., and V. Ling, Imp. Adv. Oncol. (1994) 21-36).

In another embodiment, the invention relates to a method for treating viral diseases, including but not limited to hepatitis C, hepatitis B, herpes simplex virus (HSV), HIV-AIDS, poliovirus, and smallpox virus. dsRNAs of the invention are prepared as described herein to target expressed sequences of a virus, thus ameliorating viral activity and replication. The molecules can be used in the treatment and/or diagnosis of viral infected tissue, both animal and plant. Also, such molecules can be used in the treatment of virus-associated carcinoma, such as hepatocellular cancer.

In one embodiment, a pharmaceutical compositions comprising dsRNA is used to inhibit the expression of the multi-drug resistance 1 gene (“MDR1”). “Multi-drug resistance” (MDR) broadly refers to a pattern of resistance to a variety of chemotherapeutic drugs with unrelated chemical structures and different mechanisms of action. Although the etiology of MDR is multifactorial, the overexpression of P-glycoprotein (Pgp), a membrane protein that mediates the transport of MDR drugs, remains the most common alteration underlying MDR in laboratory models (Childs, S., Imp. Adv. Oncol. (1994) 21-36). Moreover, expression of Pgp has been linked to the development of MDR in human cancer, particularly in the leukemias, lymphomas, multiple myeloma, neuroblastoma, and soft tissue sarcoma (Fan., D., et al., Reversal of Multidrug Resistance in Cancer, ed. Kellen, J. A. (CRC, Boca Raton, Fla.), pp. 93-125). Recent studies showed that tumor cells expressing MDR-associated protein (MRP) (Cole, S. P. C., et al., Science (1992) 258:1650-1654) and lung resistance protein (LRP) (Scheffer, G. L., et al., Nat. Med. (1995)1:578-582) and mutation of DNA topoisomerase II (Beck, W. T., J. Natl. Cancer Inst. (1989) 81:1683-1685) also may render MDR.

In one embodiment, the comprises administering a pharmaceutical composition comprising a dsRNA, wherein the dsRNA comprises a nucleotide sequence which is complementary to at least a part of an RNA transcript of the target gene of the mammal to be treated. The RNA transcript may be a primary or processed RNA transcript. The nucleotide sequences may be 19 to 24 nucleotides in length, preferably 20 to 24 nucleotides in length, more preferably 21 to 23 nucleotides in length, even more preferably 22 nucleotides in length, and most preferably 23 nucleotides in length.

The pharmaceutical compositions encompassed by the invention may be administered by any means known in the art including, but not limited to oral or parenteral routes, including intravenous, intramuscular, intraperitoneal, subcutaneous, transdermal, airway (aerosol), rectal, vaginal and topical (including buccal and sublingual) administration. In preferred embodiments, the pharmaceutical compositions are administered by intravenous or intraparenteral infusion or injection.

IV. Methods for Inhibiting Expression of a Target Gene.

In yet another aspect, the invention relates to a method for inhibiting the expression of a target gene in a mammal. The method comprises administering a pharmaceutical composition of the invention to a mammal, such as a human, such that expression of the target gene is silenced. Because of their surprisingly improved specificity, the dsRNAs of the present invention specifically target RNAs (primary or processed) of target genes, and at surprisingly low dosages. Compositions and methods for inhibiting the expression of a target gene using dsRNAs can be performed as described in the preceding sections, which are hereby incorporated by reference.

In one embodiment, the comprises administering a pharmaceutical composition comprising a dsRNA, wherein the dsRNA comprises a nucleotide sequence which is complementary to at least a part of an RNA transcript of the target gene of the mammal to be treated. The RNA transcript may be a primary or processed RNA transcript. The nucleotide sequences may be 19 to 24 nucleotides in length, preferably 20 to 24 nucleotides in length, more preferably 21 to 23 nucleotides in length, even more preferably 22 nucleotides in length, and most preferably 23 nucleotides in length.

The pharmaceutical compositions encompassed by the invention may be administered by any means known in the art including, but not limited to oral or parenteral routes, including intravenous, intramuscular, intraperitoneal, subcutaneous, transdermal, airway (aerosol), rectal, vaginal and topical (including buccal and sublingual) administration. In preferred embodiments, the pharmaceutical compositions are administered by intravenous or intraparenteral infusion or injection.

The methods for inhibition the expression of a target gene can be applied to any mammalian gene one wishes to silence, thereby specifically inhibiting its expression. Examples of genes which can be targeted for silencing include, without limitation, an oncogene; cytokinin gene; idiotype protein gene (Id protein gene); prion gene; gene that expresses molecules that induce angiogenesis, adhesion molecules, and cell surface receptors; genes of proteins that are involved in metastasizing and/or invasive processes; genes of proteases as well as of molecules that regulate apoptosis and the cell cycle; genes that express the EGF receptor; the multi-drug resistance 1 gene (MDR1 gene); a gene or component of a virus, particularly a human pathogenic virus, that is expressed in pathogenic organisms, preferably in plasmodia.

The present invention is illustrated by the following examples, which are not intended to be limiting in any way.

### EXAMPLES

### Example 1

### RNA Interference in a Mouse Model

In this Example, double stranded siRNAs are used to inhibit GFP gene expression in transgenic mice.

Synthesis and Preparation of dsRNAs

Oligoribonucleotides are synthesized with an RNA synthesizer (Expedite 8909, Applied Biosystems, Weiterstadt, Germany) and purified by High Pressure Liquid Chromatography (HPLC) using NucleoPac PA-100 columns, 9×250 mm (Dionex Corp.; low salt buffer: 20 mM Tris, 10 mM NaClO₄, pH 6.8, 10% acetonitrile; the high-salt buffer was: 20 mM Tris, 400 mM NaClO4, pH 6.8, 10% acetonitrile. flow rate: 3 ml/min). Formation of double stranded siRNAs is then achieved by heating a stoichiometric mixture of the individual complementary strands (10 M) in 10 mM sodium phosphate buffer, pH 6.8, 100 mM NaCl, to 80-90° C., with subsequent slow cooling to room temperature over 6 hours,

In addition, dsRNA molecules with linkers may be produced by solid phase synthesis and addition of hexaethylene glycol as a non-nucleotide linker (D. Jeremy Williams, Kathleen B. Hall, Biochemistry, 1996, 35, 14665-14670). A Hexaethylene glycol linker phosphoramidite (Chruachem Ltd, Todd Campus, West of Scotland Science Park, Acre Road, Glasgow, G20 OUA, Scotland, UK) is coupled to the support bound oligoribonucleotide employing the same synthetic cycle as for standard nucleoside phosphoramidites (Proligo Biochemie GmbH, Georg-Hyken-Str. 14, Hamburg, Germany) but with prolonged coupling times. Incorporation of linker phosphoramidite is comparable to the incorporation of nucleoside phosphoramidites.

| | | | | |
|---|---|---|---|---|
| | | | Nucleotide number | |
| | | | (overhang at the 3′- | |
| | | | end of the S1 | |
| | Sequence | | double-stranded | |
| | protocol | | region-overhang | |
| Name | No. | DsRNA sequence | | at the 3′-end of S2)] |

| | | | |
|---|---|---|---|
| S1 | 1 | (S2) 5′-CCACAUGAAGCAGCACGACUUC-3′ | |
| | 2 | (S1) 3′-GGUGUACUUCGUCGUGCUGAAG-5′ | 0-22-0 |
| S7 | 3 | (S2) 5′-CCACAUGAAGCAGCACGACUU-3′ | |
| | 4 | (S1) 3′-CUGGUGUACUUCGUCGUGCUG-5′ | 2-19-2 |
| K1 | 5 | (S2) 5′-ACAGGAUGAGGAUCGUUUCGCA-3′ | |
| | 6 | (S1) 3′-UGUCCUACUCCUAGCAAAGCGU-5′ | 0-22-0 |
| K3 | 7 | (S2) 5′-GAUGAGGAUCGUUUCGCAUGA-3′ | |
| | 8 | (S1) 3′-UCCUACUCCUAGCAAAGCGUA-5′ | 2-19-2 |
| K4 | 9 | (S2) 5′-GAUGAGGAUCGIJUUCGCAUGA-3′ | |
| | 10 | (S1) 3′-UCCUACUCCUAGCAAAGCGUACU-5′ | 2-21-0 |
| S7/S11 | 11 | (S2) 5′-CCACAUGAAGCAGCACGACUU-3′ | 2-21-0 |
| 12 | (S1) 3′-CUGGUGUACUUCGUCGUGCUGAA-5′ | | |

RNAi Administration

DsRNA are administered systemically either orally, by means of inhalation, infusion, or injection, preferably by intravenous or intraperitoneal infusion or injection in combination with pharmaceutically acceptable carriers. Examples of suitable carriers are found in standard pharmaceutical texts, e.g. “Remington's Pharmaceutical Sciences”, 16th edition, Mack Publishing Company, Easton, Pa., 1980. A preparation that is suitable for inhalation, infusion, or injection preferably consists of dsRNA and a physiologically tolerated solvent, preferably a physiological saline solution or a physiologically tolerated buffer, preferably a phosphate buffered saline solution. The invention anticipates the use of a double-stranded ribonucleic acid in a dosage of a maximum of 5 mg/kg body weight per day.

GFP Laboratory Mice:

The transgenic laboratory mouse strain TgN (GFPU) 5Nagy (Jackson Laboratory, Bar Harbor, Me.), which expresses GFP in all cells studied to date (with the help of a beta actin promoter and a CMV intermediate early enhancer) (Hadjantonakis A K et al., 1998, Nature Genetics 19: 220-222), was used. The GFP transgenic mice may be clearly differentiated on the basis of fluorescence (using a UV lamp) from the corresponding wild types (WT). The following experiments were carried out using GFP-heterozygote animals that were bred by mating a WT animal each with a heterozygote GFP-type animal. The animals were kept under controlled conditions in groups of 3-5 animals in Type III Makrolon cages (Ehret Co., Emmendingen, Germany) at a constant temperature of 22° C. and a light-to-dark rhythm of 12 hours. Granulated softwood (8/15, Altromin Co., Lage, Germany) was strewn on the bottom of the cages. The animals received tap water and Altromin 1324 pelleted standard feed (Altromin Co.) ad libitum.

In Vivo Experiment:

Heterozygote GFP animals were placed in cages as described above in groups of 3. DsRNA solution was injected intravenously (i.v.) into the caudal vein in 12-hour rotation (between 5:30 and 7:00 and between 17:30 and 19:00) over 5 days. Injection volume was 60 μl per 10 g body weight, and dosage was 2.5 mg dsRNA or 50 μg per kg body weight. The groups were organized as follows:

Group A: PBS (phosphate buffered saline) 60 μl per 10 g body weight each,

Group B: 2.5 mg per kg body weight of a non-specific control dsRNA (K1 control with smooth ends and a double-stranded region of 22 nucleotide pairs),

Group C: 2.5 mg per kg body weight of another non-specific control dsRNA (K3 control with 2 nucleotide [nt] overhangs and both 3′-ends and a double-stranded region of 19 nucleotide pairs),

Group D: 2.5 mg per kg body weight of dsRNA (directed specifically against GFP, henceforth designated as S1, with smooth ends and a double-stranded region of 22 nucleotide pairs),

Group E: 2.5 mg dsRNA per kg body weight (directed specifically against GFP, henceforth designated as S7, with 2 nt overhangs and the 3′-ends of both strands, and a double-stranded region of 19 nucleotide pairs),

Group F: 50 μg S1 dsRNA per kg body weight (in other words 1/50 the dosage of Group D).

After the last injection of a series of 10 injections, the animals were sacrificed after 14-20 hours, and the organs and blood were removed as described below.

Organ Removal:

Immediately after the animals were killed by C02 inhalation, the blood and various organs were removed (thymus, lungs, heart, spleen, stomach, intestines, pancreas, brain, kidneys, and liver). The organs were quickly rinsed in cold sterile PBS and dissected with a sterile scalpel. A portion was fixed for 24 hours for immunohistochemical staining in methyl Carnoy (MC, 60% methanol, 30% chloroform, 10% glacial acetic acid); another portion was immediately flash-frozen in liquid nitrogen for freeze sections and protein isolation, and stored at −80° C.; and another smaller portion was frozen for RNA isolation at −80° C. in RNAeasy Protect (QIAGEN GmbH, Max Volmer Str. 4, 40724 Hilden). Immediately after removal, the blood was kept on ice for 30 minutes, mixed, centrifuged for 5 minutes at 2000 rpm (Mini Spin, Eppendorf AG, Barkhausenweg 1, 22331, Hamburg, Germany), and the supernatant fluid was drawn off and stored at −80° C. (designated here as plasma).

Processing the Biopsies:

After fixing the tissue for 24 hours in MC, the tissue pieces were dehydrated in an ascending alcohol series at room temperature: 40 minutes each 70% methanol, 80% methanol, 2×96% methanol and 3×100% isopropanol. After that the tissue was warmed up in 100% isopropanol at 60° C. in an incubator, after which it was incubated for 1 hour in an isopropanol/paraffin mixture at 60° C. and 3 x for 2 hours in paraffin, and then embedded in paraffin. Tissue sections 3 μm in thickness were prepared for immunoperoxidase staining, using a rotation microtome (Leica Microsystems Nussloch GmbH, Heidelberger Str. 17-19, 69226 Nussloch, Germany), placed on microscopic slides (Superfrost, Vogel GmbH & Co. KG, Medical Technology and Electronics, Marburger Str. 81, 35396 Giessen, Germany), and incubated for 30 minutes at 60° C.

Immunoperoxidase Staining for GFP:

The sections were deparaffinized for 3×5 minutes in xylol, rehydrated in a descending alcohol series (3×3 min. 100% ethanol, 2×2 min. 95% ethanol), and then incubated for 20 minutes in 3% H202/methanol to block endogenous peroxidases. Next, all incubation steps were carried out in a moist chamber. After 3×3 min. washing with PBS, the sections were incubated with a first antibody (goat anti-GFP antibody, sc-5384, Santa Cruz Biotechnology, Inc., Berheimer Str. 89-2, 69115 Heidelberg, Germany) 1:500 in 1% BSA/PBS overnight at 4° C. The sections were then incubated with the biotinylated secondary antibody (donkey anti-goat IgG; Santa Cruz Biotechnology; 1:2000 dilution) for 30 minutes at room temperature, after which they were incubated for 30 minutes with Avidin D peroxidase (1:2000 dilution, Vector Laboratories, 30 Ingold Road, Burlingame, Calif. 94010). After each antibody incubation, the sections were washed in PBS for 3×3 min., and buffer residue was removed from the sections along with cell material. All antibodies were diluted with 1% bovine serum albumin (BSA)/PBS. The sections were stained with 3,3′-diamino benzidine (DAB) using the DAB Substrate Kit (Vector Laboratories) in accordance with the manufacturer's instructions. Gill's Hematoxylin III (Merck KgaA, Frankfurter Str. 250, 64293 Darmstadt) was used as the nuclear counterstain. After dehydration in an ascending alcohol series and 3×5 minutes xylol, the sections were covered with Entellan (Merck). Microscopic evaluation of the stains was accomplished using a IX50 microscope from OLYMPUS Optical Co. (Europe) GmbH, Wendenstr. 14-18 20097 Hamburg, Germany, fitted with a CCD camera (Hamamatsu Photonics K.K., Systems Division, 8012 Joko-cho Hamamatsu City, 431-3196 Japan).

Protein Isolation From Tissue Pieces:

Frozen tissue samples were added to 800 μl isolation buffer (50 m HEPES, pH 7.5; 150 mM NaCl; 1 mM EDTA; 2.5 mM EGTA; 10% glycerol; 0.1% Tween; 1 mM DTT; 10 mM β-glycerol phosphate; 1 mM NaF; 0.1 mM Na3VO4 with a “complete” protease inhibitor tablet from Roche Diagnostics GmbH, Roche Applied Science, Sandhofer Str. 116, 68305 Mannheim), and homogenized for 2×30 seconds with an ultraturrax (DIAX 900, Dispersion Tool 6G, HEIDOLPH Instruments GmbH & Co. KG, Walpersdorfer Str. 12, 91126 Schwabach), and cooled on ice in between steps. After incubation for 30 minutes on ice, the homogenate was mixed and centrifuged for 20 minutes at 10,000 g, 4° C. (3K30, SIGMA Laboratory Centrifuge GmbH, An der Unteren Söse 50,37507 Osterode am Harz). The supernatant fluid was again incubated for 10 minutes on ice, mixed, and centrifuged for 20 minutes at 15,000 g, 4° C. Protein determination of the supernatant fluid was determined according to Bradford, 1976, modified according to Zor & Selinger, 1996, using the Roti-Nanoquant system (Carl Roth GmbH & Co., Schoemperlenstr. 1-5, 76185 Karlsruhe, Germany) in accordance with manufacturer's instructions. BSA was used for protein calibration in a concentration range of 10 to 100 μg/ml.

SDS Gel Electrophoresis:

Denaturing, discontinuous 15% SDS-PAGE (polyacrylamide gel electrophoresis) according to Läemmli (Nature 277: 680-685, 1970) was carried out in a Multigel-Long electrophoresis chamber (Whatman Biometra GmbH, Rudolf Wissell Str. 30, 37079 Göttingen). The separation gel was poured on to a thickness of 1.5 mm: 7.5 ml acrylamide/bisacrylamide (30%, 0.9%); 3.8 ml 1.5 M Tris/HCl, pH 8.4; 150 μl 10% SDS; 3.3 ml distilled water; 250 μl ammonium persulfate (10%); 9 μl TEMED (N,N,N′,N′-tetramethylendiamine), and covered over with 0.1% SDS until polymerization occurred. A collection gel was then poured on: 0.83 μl acrylamide/bisacrylamide (30%, 0.9%), 630 μl 1 M tris/HCl, pH 6.8; 3.4 ml distilled water; 50 μl 10% SDS; 50 μl 10% ammonium persulfate; 5 μl TEMED.

A corresponding quantity of 4× sample buffer (200 mM Tris, pH 6.8, 4% SDS, 100 mM DTT (dithiotreithol), 0.02% bromophenol blue, 20% glycerin) was then added to the proteins, which were then denatured on a heat block at 100° C., centrifuged on ice after cooling off, and then applied to the gel. The same plasma and protein quantities were used in each lane (3 μl plasma or 25 μg total protein each). Protein electrophoresis was carried out at room temperature at a constant 50V. The protein gel marker Kaleidoscope Prestained Standard (Bio-Rad Laboratories GmbH, Heidemannstr. 164, 80939 Munich) was used as molecular marker.

Western Blot and Immunodetection:

Proteins separated by SDS-PAGE were transferred to a PVDF (polyvinyl difluoride) membrane (Hybond-P, Amersham Biosciences Europe GmbH, Munzinger Str. 9, 79111 Freiburg, Germany) using the semidry transfer method according to Kyhse-Anderson (J. Biochem. Biophys. Methods 10: 203-210, 1984) at room temperature and constant amperage of 0.8 mA/cm2 for 1.5 hours in Tris/Glycerin transfer buffer (39 mM glycerin, 46 mM tris, 0.1% SDS, and 20% methanol). After immunodetection both the gels and the blots, as well as the blot membranes, were stained with Coomassie (0.1% Coomassie G250, 45% methanol, 10% glacial acetic acid) in order to check for electrophoretic transfer. The blot membranes were incubated after transfer in 1% skim milk powder/PBS for 1 hour at room temperature to saturate nonspecific bonds. Next, each membrane was washed three times for 3 minutes with 0.1% Tween-20/PBS. All subsequent antibody incubations and wash steps were done in 0.1% Tween-20/PBS. The primary antibody (goat anti-GFP antibody, sc-5384, Santa Cruz Biotechnology) was incubated for one hour at room temperature at a dilution of 1:1000. After washing 3×5 minutes, the membranes were incubated with a horseradish peroxidase coupled secondary antibody (donkey anti-goat IgG, Santa Cruz Biotechnology), at a dilution of 1:10,000. Detection of horseradish peroxidase was then achieved using the ECL system (Amersham) in accordance with the manufacturer's instructions.

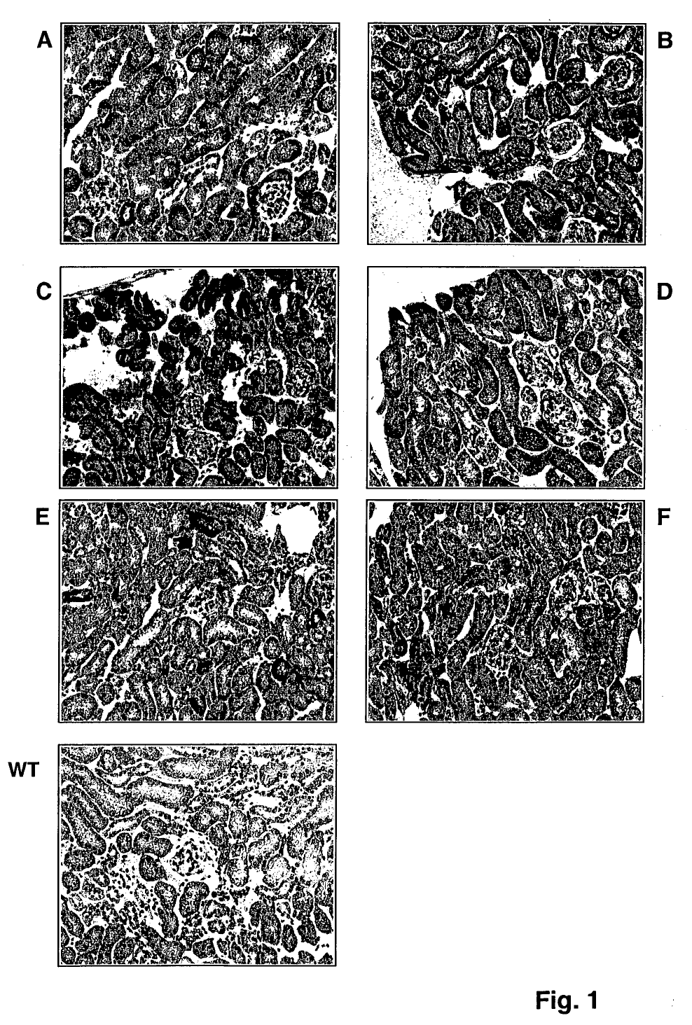

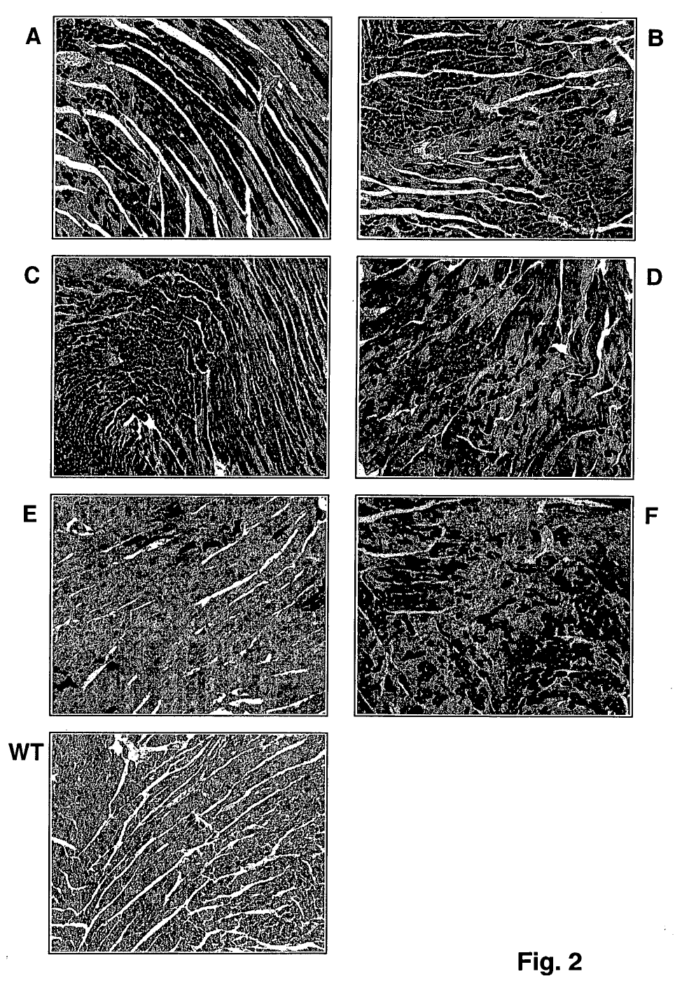

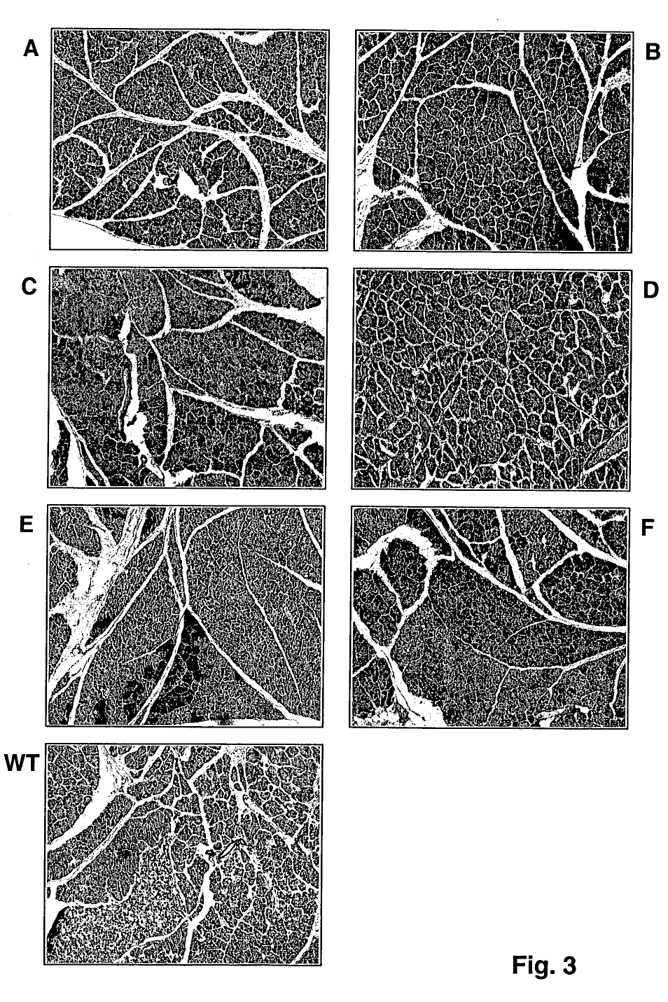

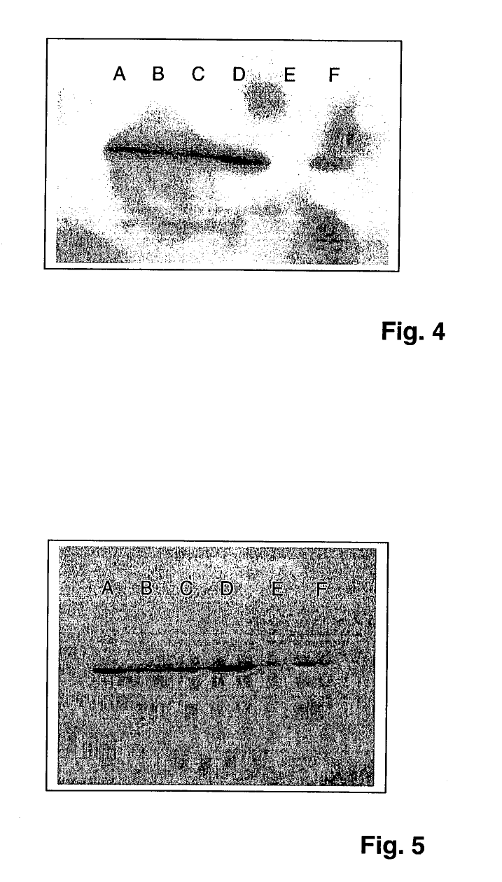

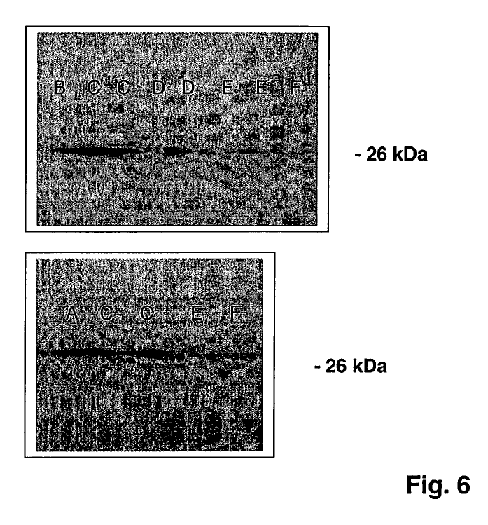

FIGS. 1 to 3 show inhibition of GFP expression after intravenous injection of specific anti-GFP dsRNA, by means of immunoperoxidase GFP staining of 3 μm paraffin sections. Over the course of the experiment, the anti-GFP dsRNA, with a double-stranded region of 22 nucleotide (nt) pairs without overhangs at the 3′-ends (D) and the corresponding non-specific control dsRNA (B), as well as the specific anti-GFP dsRNA, with a double-stranded region consisting of 19 nucleotide pairs with 2 nt overhangs at the 3′-ends (E), and the corresponding non-specific control dsRNA (C) were applied in 12-hour rotation over 5 days. (F) received 1/50 the dosage of Group (D). Animals not administered dsRNA (A) and WT animals were used as further controls. FIG. 1 shows the inhibition of GFP expression in kidney sections; FIG. 2 in heart sections; and FIG. 3 in pancreas tissue. FIGS. 4 to 6 show Western blot analyses of GFP expression in plasma and tissues. FIG. 4 shows the inhibition of GFP expression in plasma; FIG. 5 in kidney; and FIG. 6 in heart. FIG. 6 shows the total protein isolate from various animals. The same quantities of total protein were used for each track. In the animals that were given non-specific control dsRNA (animals in Groups B and C), GFP is not reduced in comparison with animals that received no dsRNA. Animals that received the specific anti-GFP dsRNA with 2 nt overhangs at the 3′-ends of both strands and a double-stranded region consisting of 19 nucleotide pairs showed significantly inhibited GFP expression in the tissues studied (heart, kidneys, pancreas, and blood), compared with untreated animals (FIGS. 1-6). Of the animals in Groups D and F, who were given specific anti-GFP dsRNA, with blunt ends and a double-stranded region consisting of 22 nucleotide pairs, only those animals that received the dsRNA at a dosage of 50 μg/kg body weight per day demonstrated specific inhibition of GFP expression. However, the degree of inhibition was less marked than that seen with the animals in Group E.

A summary evaluation of GFP expression in tissue sections and Western blot shows that the inhibition of GFP expression is greatest in blood and in kidneys (FIGS. 1, 4, and 5).

### Example 2

### Efficacy of RNA Interference in a Mouse Model

An additional study examined whether the dosage of 5 mg/kg body weight (BW) per day, which was shown to be effective in the first experiment, could be further reduced. To this end, a dosage 200 times weaker, i.e., 25 μg/kg BW per day, was intravenously injected into the caudal vein of transgenic GFP mice. The GFP-specific dsRNA construct S7/S11, which is derived from the GFP sequence and was shown to be particularly effective in in vitro transcription assays, was used. The non-specific control dsRNA, K4, exhibits the same construction as S7/S11 (dsRNA with 21 base pairs and a 2 nt overhangs at the 3′-end of the S1 antisense strand) but is derived from the 5′-end of the neomycin resistance gene.

In order to study the effectiveness of GFP-specific dsRNA, the percentage of GFP-positive lymphocytes in the blood was tested after the end of the experiment, using FACS analysis (fluorescence activated cell sorting), as was GFP expression in total blood, using Western blot analysis.

During the experiment, the heterozygote GFP test animals were kept in cages in groups of 2 to 3 animals as described above. The injections were administered intravenously to the caudal vein over a span of 21 days once per day in the morning without anesthesia. Injection volume was 60 μl per 10 g BW, and the dosage was 25 μg dsRNA (GFP-specific dsRNA) and 250 μg dsRNA (K4 non-specific control dsRNA), respectively. The test animals were divided into two groups:

The GFP group consisted of 7 animals, which received 25 μg/kg body weight of the GFP-specific S7/S11 dsRNA. The control group, consisting of 6 animals, received the non-specific K4 control dsRNA at a concentration of 250 μg/kg body weight. The last injection was administered on Day 21. Exactly 24 hours after the final injection i.e. day 22, the animals were sacrificed with CO2 and the abdominal cavity was opened, and blood was immediately drawn off by means of cardiopuncture with a syringe. Approximately 100 μl whole blood were flash-frozen in liquid nitrogen without further treatment for Western blot analysis. In order to inhibit coagulation, 100 mM of sodium citrate was added 1:1 to the largest portion of the blood, carefully mixed, and stored in the dark at room temperature for FACS analysis.

FACS Analysis:

Erythrolysis, which was automated using the Immunoprep Reagent Kit (Beckman Coulter GmbH-Diagnostics, Siemensstr. 1, 85716 Unterschleissheim, Germany) on a Coulter Q-Prep (Beckman Coulter GmbH) in accordance with manufacturer's instructions, was carried out before FACS analysis. For this, 100 μl each of the sodium citrate/blood mixture, which had been pipetted into 5 ml tubes with a round bottom, were aliquoted and the number of GFP-expressing cells was determined using a Coulter EPICS XL flow cytometer (Beckman Coulter GmbH). Quantitative analysis of the B- and T-cells, as well as of the granulocytes, macrophages, and monocytes, was determined by means of direct staining and subsequent FACS analysis. The following phycoerithrin-marked monoclonal antibodies were used in the analysis:

Rat anti-mouse CD19 (Clone 1D3) as the marker for B-lymphocytes,

CD11 (Clone M1/70) as the marker for granulocytes, macrophages, and monocytes,

Rat anti-mouse CD3 (Clone 17A2 has the marker for T-lymphocytes,

and to further differentiate the T-cells: CD4 (Clone GK1.5) as the marker for natural killer T-cells, and CD8a (Clone 53-6.7) as the marker for cytotoxic T-cells.

All antibodies were obtained from BD BioSciences, Tullastr. 8-12, 69126 Heidelberg, Germany. Staining with the corresponding antibodies was carried out before the erythrolysis described above. For this, 10 μl antibodies each were placed in 5-ml FACS tubes, 100 μl blood was added and then incubated at room temperature in the dark for 30 minutes. After erythrolysis, a 2-color fluorescence measurement was taken (stimulus wavelength: 488 nm). The blood from two completely untreated GFP animals was analyzed as a control. The percentages of GFP expression for each individual animal is thus the average of 6 individual measurements (one 1-color fluorescence measurement without staining, and 5 each of 2-color fluorescence measurements with antibody staining).

SDS Gel Electrophoresis:

Denaturing, discontinuous 15% SDS-PAGE (polyacrylamide gel electrophoresis) according to Läemmli (Nature 277: 680-685, 1970) was carried out in a Multigel-Long electrophoresis chamber (Whatman Biometra GmbH, Rudolf Wissell Str. 30, 37079 Göttingen). The separation gel was poured on to a thickness of 1.5 mm: 7.5 ml acrylamide/bisacrylamide (30%, 0.9%); 3.8 ml 1.5 M Tris/HCl, pH 8.4; 150 μl 10% SDS; 3.3 ml distilled water; 250 μl ammonium persulfate (10%); 9 μl TEMED (N,N,N′,N′-tetramethylendiamine), and covered over with 0.1% SDS until polymerization occurred. A collection gel was then poured on: 0.83 μl acrylamide/bisacrylamide (30%, 0.9%), 630 μl 1 M tris/HCl, pH 6.8; 3.4 ml distilled water; 50 μl 10% SDS; 50 μl 10% ammonium persulfate; 5 μl TEMED.

Before being applied to the gel, the whole blood was lysed with ultrasound after which 4× sample buffer (200 mM tris, pH 6.8, 4% SDS, 100 mM DTT (dithiotreithol), 0.02% bromophenol blue, 20% glycerin) was added prior to denaturation on a heat block at 100° C., cooling on ice, brief centrigugation and loading on to the gel 2 μl whole blood/lane). Water-cooled electrophoresis was carried at room temperature at a constant 50V. Kaleidoscope Prestained Standard protein gel marker (Bio-Rad) served as molecular standard.

Western Blot:

Proteins separated by SDS-PAGE were then transferred to PVDF (polyvinyl difluoride) membrane (Hybond-P, Amersham) using the semidry transfer method according to Kyhse-Anderson (J. Biochem. Biophys. Methods 10: 203-210, 1984) at room temperature and constant amperage of 0.8 mA/cm2 for 1.5 hours in Tris/Glycerin transfer buffer (39 mM glycerin, 46 mM tris, 0.1% SDS, and 20% methanol). After immunodetection both the gels and the blots, as well as the blot membranes were stained with Coomassie (0.1% Coomassie G250, 45% methanol, 10% glacial acetic acid) in order to check for electrophoretic transfer. The blot membranes were incubated after transfer in 1% skim milk powder/PBS for 1 hour at room temperature to saturate nonspecific bonds and then washed three times for 3 minutes with 0.1% Tween-20/PBS. All subsequent antibody incubations and wash steps were done in 0.1% Tween-20/PBS. The primary antibody (goat anti-GFP antibody, sc-5384, Santa Cruz Biotechnology) was incubated for one hour at room temperature at a dilution of 1:1000. After washing for 3×5 minutes, the membranes were incubated with horseradish peroxidase coupled secondary antibody (donkey anti-goat IgG, Santa Cruz Biotechnology), at a dilution of 1:10,000. Detection of horseradish peroxidase was then achieved using the ECL system (Amersham) in accordance with the manufacturer's instructions.

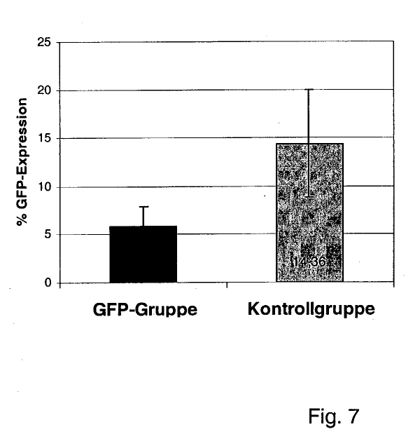

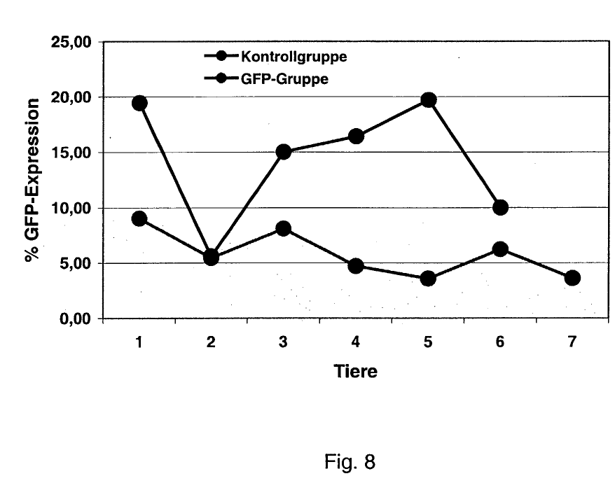

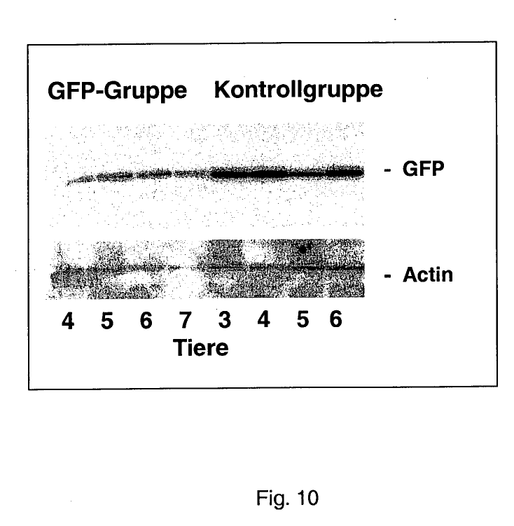

FIGS. 7, 8, and 10, show the inhibition of GFP expression, analyzed by means of FACS, in lymphocytes (FIGS. 7 and 8), and in whole blood, analyzed by Western blot, after injection of specific anti-GFP dsRNA (FIG. 10). The values shown in FIG. 7 correspond to the average values of those shown in FIG. 8. The application of 25 μg in GFP-specific dsRNA per kg body weight per day in the GFP group led to a significant and specific reduction in GFP expression, when compared to the control group, which received 250 μg of a nonspecific control dsRNA per kg body weight per day over the course of the experiment. The dsRNA concentrations were here markedly less than those used in the first in vivo experiment. In the first in vivo experiment, injections were administered over 10 days in a 12-hour rhythm, so that a total daily dosage of 5 mg dsRNA per kg body weight was reached. In comparison, the total daily dosage in the second in vivo experiment described here was 25 μg dsRNA per kg body weight (the injections were given once per day over 21 days). This total daily dosage is 200 times less than that of the first in vivo experiment. The total dosage of dsRNA per kg body weight over the entire span of the experiment was 50 milligrams per kg body weight in the first in vivo experiment (2.5 mg/kg body weight x 20 injections) and in the second in vivo experiment, 0.525 mg per kg body weight (25 μg/kg body weight x 21 injections). This corresponds approximately to a quantity of dsRNA that is 95 times less. However, the reduction in GFP expression in the blood is comparable in both studies.

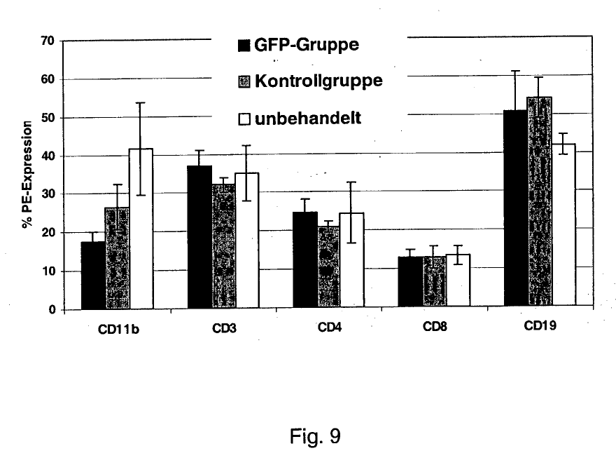

FIG. 9 shows that application of the specified dsRNAs leads to no change in blood composition over a span of 21 days. Therefore, the reduction in GFP expression in the group treated with GFP-specific dsRNA is not the result of a decrease in GFP expressing blood cells.

### Example 3

### Treatment of a CML Patient with BCR-ABL siRNA

In this Example, Bcr-Abl-specific double stranded siRNA is injected into CML patients and shown to specifically inhibit Bcr-Abl gene expression.

SiRNA Synthesis

siRNA (BCR-ABL) directed against the fusion sequence of bcr-abl are chemically synthesized with or without a hexaethylene glycol linker as described in Example 1.

The strand complimentary to the Bcr-Abl transcript is 23 nucleotides in length whereas the strand that is not complimentary to the Bcr-Abl transcript is 21 nucleotides in length. The resulting Bcr-Abl double-stranded siRNA comprises a 2 nucleotide 3′ overhang at one end (3′ end of the complimentary strand) whereas the other end is blunt.

The sense and antisense sequences of the siRNAs are:

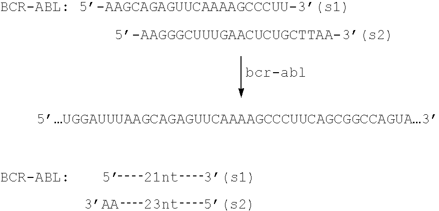

siRNA Administration and Dosage

The present example provides for pharmaceutical compositions for the treatment of human CML patients comprising a therapeutically effective amount of a BCR-ABL siRNA as disclosed herein, in combination with a pharmaceutically acceptable carrier or excipient. SiRNAs useful according to the invention may be formulated for oral or parenteral administration. The pharmaceutical compositions may be administered in any effective, convenient manner including, for instance, administration by topical, oral, anal, vaginal, intravenous, intraperitoneal, intramuscular, subcutaneous, intranasal or intradermal routes among others. One of skill in the art can readily prepare siRNAs for injection using such carriers that include, but are not limited to, saline, buffered saline, dextrose, water, glycerol, ethanol, and combinations thereof. Additional examples of suitable carriers are found in standard pharmaceutical texts, e.g. “Remington's Pharmaceutical Sciences”, 16th edition, Mack Publishing Company, Easton, Pa., 1980.

The dosage of the siRNAs will vary depending on the form of administration. In the case of an injection, the therapeutically effective dose of siRNA per injection is in a dosage range of approximately 1-500 milligram/kg body weight, preferably 25 microgram/kg body weight. In addition to the active ingredient, the compositions usually also contain suitable buffers, for example phosphate buffer, to maintain an appropriate pH and sodium chloride, glucose or mannitol to make the solution isotonic. The administering physician will determine the daily dosage which will be most suitable for an individual and will vary with the age, gender, weight and response of the particular individual, as well as the severity of the patient's symptoms. The above dosages are exemplary of the average case. There can, of course, be individual instances where higher or lower dosage ranges are merited, and such are within the scope of this invention. The siRNAs of the present invention may be administered alone or with additional siRNA species or in combination with other pharmaceuticals.

RNA purification and Analysis

Efficacy of the siRNA treatment is determined at defined intervals after the initiation of treatment using real time PCR on total RNA extracted from peripheral blood.

Cytoplasmic RNA from whole blood, taken prior to and during treatment, is purified with the help of the RNeasy Kit (Qiagen, Hilden) and Bcr-abl mRNA levels are quantitated by real time RT-PCR.

Real Time PCR Analysis

Real-time Taqman-RT-PCR is performed as described previously (Eder M et al. Leukemia 1999; 13: 1383-1389; Scherr M et al. BioTechniques. 2001; 31: 520-526).

The probes and primers are:

| | |
|---|---|
| bcrFP: | 5′-AGCACGGACAGACTCATGGG-3′, |
| bcrRP: | 5′-GCTGCCAGTCTCTGTCCTGC-3′, |

ber—Taqman—Probe:

| | |
|---|---|
| bcr-Taqman-probe: | 5′-AGGGCCAGGTCCAGCTGGACCC-3′ |
| ablFP: | 5′-GGCTGTCCTCGTCCTCCAG-3′, |
| ablRP: | 5′-TCAGACCCTGAGGCTCAAAGT-3′, |

abl—Taqman—Probe:

abl-Taqman-probe:
5′-ATCTGGAAGAAGCCCTTCAGCGGC-3′

Bcr-abl RNA levels in peripheral blood from CML patients treated with BCR-ABL siRNAs or control siRNAs (with or without hexaethylene glycol linker) are determined by real time RT-PCR and standardized against an internal control e.g. GAPDH mRNA levels. Analysis by real time PCR at regular intervals, for example every 12-24 hours, provides the attending physician with a rapid and accurate assessment of treatment efficacy as well as the opportunity to modify the treatment regimen in response to the patient's symptoms and disease progression.

It will be evident to a person of skill in the art how the siRNA treatment described above can be adapted to the treatment for any disease for which the genes causing the disease are known, as disclosed herein.

### Example 4

### siRNA Expression Vectors

In another aspect of the invention, siRNA molecules that interact with target RNA molecules and modulate gene expression activity are expressed from transcription units inserted into DNA or RNA vectors (see for example Couture et A, 1996, TIG., 12, 5 1 0, Skillern et A, International PCT Publication No. WO 00/22113, Conrad, International PCT Publication No. WO 00/22114, and Conrad, U.S. Pat. No. 6,054,299). These transgenes can be introduced as a linear construct, a circular plasmid, or a viral vector, which can be incorporated and inherited as a transgene integrated into the host genome. The transgene can also be constructed to permit it to be inherited as an extrachromosomal plasmid (Gassmann et al., 1995, Proc. Natl. Acad. Sci. USA 92:1292).

The individual strands of a siRNA can be transcribed by promoters on two separate expression vectors and cotransfected into a target cell. Alternatively each individual strand of the siRNA can be transcribed by promoters both of which are located on the same expression plasmid. In a preferred embodiment, the siRNA is expressed as an inverted repeat joined by a linker polynucleotide sequence such that the siRNA has a stem and loop structure.

The recombinant siRNA expression vectors are preferably DNA plasmids or viral vectors. siRNA expressing viral vectors can be constructed based on, but not limited to, adeno-associated virus (for a review, see Muzyczka et al. (1992, Curr. Topics in Micro. and Immunol. 158:97-129)), adenovirus (see, for example, Berkner et al. (1988, BioTechniques 6:616), Rosenfeld et al. (1991, Science 252:431-434), and Rosenfeld et al. (1992, Cell 68:143-155)), or alphavirus as well as others known in the art. Retroviruses have been used to introduce a variety of genes into many different cell types, including epithelial cells, in vitro and/or in vivo (see for example Eglitis, et al., 1985, Science 230:1395-1398; Danos and Mulligan, 1988, Proc. Natl. Acad. Sci. USA 85:6460-6464; Wilson et al., 1988, Proc. Natl. Acad. Sci. USA 85:3014-3018; Armentano et al., 1990, Proc. Natl. Acad. Sci. USA 87:61416145; Huber et al., 1991, Proc. Natl. Acad. Sci. USA 88:8039-8043; Ferry et al., 1991, Proc. Natl. Acad. Sci. USA 88:8377-8381; Chowdhury et al., 1991, Science 254:1802-1805; van Beusechem. et al., 1992, Proc. Nad. Acad. Sci. USA 89:7640-19; Kay et al., 1992, Human Gene Therapy 3:641-647; Dai et al., 1992, Proc. Natl. Acad. Sci. USA 89:10892-10895; Hwu et al., 1993, J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573). Recombinant retroviral vectors capable of transducing and expressing genes inserted into the genome of a cell can be produced by transfecting the recombinant retroviral genome into suitable packaging cell lines such as PA317 and Psi-CRIP (Comette et al., 1991, Human Gene Therapy 2:5-10; Cone et al., 1984, Proc. Natl. Acad. Sci. USA 81:6349). Recombinant adenoviral vectors can be used to infect a wide variety of cells and tissues in susceptible hosts (e.g., rat, hamster, dog, and chimpanzee) (Hsu et al., 1992, J. Infectious Disease, 166:769), and also have the advantage of not requiring mitotically active cells for infection.

The promoter driving siRNA expression in either a DNA plasmid or viral vector of the invention may be a eukaryotic RNA polymerase I (e.g. ribosomal RNA promoter), RNA polymerase II (e.g. CMV early promoter or actin promoter or U1 snRNA promoter) or preferably RNA polymerase III promoter (e.g. U6 snRNA or 7SK RNA promoter) or a prokaryotic promoter, for example the T7 promoter, provided the expression plasmid also encodes T7 RNA polymerase required for transcription from a T7 promoter. The promoter can also direct transgene expression to specific organs or cell types (see, e.g., Lasko et al., 1992, Proc. Natl. Acad. Sci. USA 89:6232). Several tissue-specific regulatory sequences are known in the art including the albumin regulatory sequence for liver (Pinkert et al., 1987, Genes Dev. 1:268276); the endothelin regulatory sequence for endothelial cells (Lee, 1990, J. Biol. Chem. 265:10446-50); the keratin regulatory sequence for epidennis; the myosin light chain-2 regulatory sequence for heart (Lee et al., 1992, J. Biol. Chem. 267:15875-85), and the insulin regulatory sequence for pancreas (Bucchini et al., 1986, Proc. Natl. Acad. Sci. USA 83:2511-2515), or the vav regulatory sequence for hematopoietic cells (Oligvy et al., 1999, Proc. Natl. Acad. Sci. USA 96:14943-14948). Another suitable regulatory sequence, which directs constitutive expression of transgenes in cells of hematopoietic origin, is the murine MHC class I regulatory sequence (Morello et al., 1986, EMBO J. 5:1877-1882). Since NMC expression is induced by cytokines, expression of a test gene operably linked to this promoter can be upregulated in the presence of cytokines.

In addition, expression of the transgene can be precisely regulated, for example, by using an inducible regulatory sequence and expression systems such as a regulatory sequence that is sensitive to certain physiological regulators, e.g., circulating glucose levels, or hormones (Docherty et al., 1994, FASEB J. 8:20-24). Such inducible expression systems, suitable for the control of transgene expression in cells or in mammals include regulation by ecdysone, by estrogen, progesterone, tetracycline, chemical inducers of dimerization, and isopropyl-beta-D1-thiogalactopyranoside (EPTG). A person skilled in the art would be able to choose the appropriate regulatory/promoter sequence based on the intended use of the siRNA transgene.

Preferably, recombinant vectors capable of expressing siRNA molecules are delivered as described below, and persist in target cells. Alternatively, viral vectors can be used that provide for transient expression of siRNA molecules. Such vectors can be repeatedly administered as necessary. Once expressed, the siRNAs bind to target RNA and modulate its function or expression. Delivery of siRNA expressing vectors can be systemic, such as by intravenous or intramuscular administration, by administration to target cells ex-planted from the patient followed by reintroduction into the patient, or by any other means that allows for introduction into a desired target cell.

SiRNA expression DNA plasmids are typically transfected into target cells as a complex with cationic lipid carriers (e.g. Oligofectamine) or non-cationic lipid-based carriers (e.g. Transit-TKO™). Multiple lipid transfections for siRNA-mediated knockdowns targeting different regions of a single target gene or multiple target genes over a period of a week or more are also contemplated by the present invention. Successful introduction of the vectors of the invention into host cells can be monitored using various known methods. For example, transient transfection, can be signaled with a reporter, such as a fluorescent marker, such as Green Fluorescent Protein (GFP). Stable transfection of ex vivo cells can be ensured using markers that provide the transfected cell with resistance to specific environmental factors (e.g., antibiotics and drugs), such as hygromycin B resistance.

For a review of techniques that can be used to generate and assess transgenic animals, skilled artisans can consult Gordon (IwL Rev. CytoL 1 1 5:171-229, 1989), and may obtain additional guidance from, for example: Hogan et al. “Manipulating the Mouse Embryo” (Cold Spring Harbor Press, Cold Spring Harbor, N.Y., 1986; Krimpenfort et al., Bio/Technology 9:86, 1991; Palmiter et al., Cell 41:343, 1985; Kraemer et al., “Genetic Manipulation of the Early Mammalian Embryo,” Cold Spring Harbor Press, Cold Spring Harbor, N.Y., 1985; Hammer et al., Nature 315:680, 1985; Purcel et al., Scieizce, 244:1281, 1986; Wagner et al., U.S. Pat. No. 5,175,385; and Krimpenfort et al., U.S. Pat. No. 5,175,384.

The nucleic acid molecules of the invention described in Example 4 can also be generally inserted into vectors and used as gene therapy vectors for human patients. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

### Example 5

### Method of Determining an Effective Dose of a siRNA

A therapeutically effective amount of a composition containing a sequence that encodes an siRNA, (i.e., an effective dosage), is an amount that inhibits expression of the polypeptide encoded by the target gene by at least 10 percent. Higher percentages of inhibition, e.g., 15, 20, 30, 40, 50, 75, 85, 90 percent or higher may be preferred in certain embodiments. Exemplary doses include milligram or microgram amounts of the molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram). The compositions can be administered one time per week for between about 1 to 1 0 weeks, e.g., between 2 to 8 weeks, or between about 3 to 7 weeks, or for about 4, 5, or 6 weeks. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a composition can include a single treatment or a series of treatments. In some cases transient expression of the siRNA may be desired. When an inducible promoter is included in the construct encoding an siRNA, expression is assayed upon delivery to the subject of an appropriate dose of the substance used to induce expression.

Appropriate doses of a composition depend upon the potency of the molecule (the sequence encoding the siRNA) with respect to the expression or activity to be modulated. One or more of these molecules can be administered to an animal (e.g., a human) to modulate expression or activity of one or more target polypeptides. A physician may, for example, prescribe a relatively low dose at first, subsequently increasing the dose until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

The efficacy of treatment can be monitored either by measuring the amount of the target gene mRNA (e.g. using real time PCR) or the amount of polypeptide encoded by the target gene mRNA (Western blot analysis). In addition, the attending physician will monitor the symptoms associated with the disease or disorder afflicting the patient and compare with those symptoms recorded prior to the initiation of siRNA treatment.

We claim:

1. A pharmaceutical composition for inhibiting the expression of a target gene in a mammal, comprising a double-stranded ribonucleic acid (dsRNA) and a pharmaceutically acceptable carrier, wherein the dsRNA comprises a nucleotide sequence which is substantially identical to at least a part of the target gene and which is less than 25 nucleotides in length, and wherein the pharmaceutical composition is in a unit dosage amount of less than 5 milligram (mg) of dsRNA per kg body weight of the mammal.

2. The pharmaceutical composition of claim 1, wherein the dosage unit of dsRNA is in a range of 0.01 to 2.5 milligrams, 0.1 to 200 micrograms, 0.1 to 100 micrograms, 1.0 to 50 micrograms, or 1.0 to 25 micrograms per kilogram body weight.

3. The pharmaceutical composition of claim 1, wherein the dosage unit of dsRNA is less than 25 micrograms per kilogram body weight.

4. The pharmaceutical composition of claim 1, wherein the dsRNA further comprises a complementary RNA strand, wherein the complementary RNA strand comprises a complementary nucleotide sequence which is complementary to an mRNA transcript of a portion of the target gene, and wherein the complementary nucleotide sequence is 19 to 24 nucleotides in length, 20 to 24 nucleotides in length, or 21 to 23 nucleotides in length.

5. The pharmaceutical composition of claim 4, wherein the complementary nucleotide sequence is 22 or 23 nucleotides in length.

6. The pharmaceutical composition of claim 4, wherein the complementary RNA strand is 21 to 30 nucleotides in length, 21 to 25 nucleotides in length, or 21 to 24 nucleotides in length.

7. The pharmaceutical composition of claim 4, wherein the complementary RNA strand is 23 nucleotides in length.

8. The pharmaceutical composition of claim 1, wherein the dsRNA comprises a first complementary RNA strand and a second RNA strand, wherein the first complementary RNA strand comprises a complementary nucleotide sequence which is complementary to an RNA transcript of a portion of the target gene, and wherein the first complementary RNA strand and the second RNA strand comprise a 3′-terminus and a 5′-terminus, and wherein at least one of the first RNA and second RNA strands comprise a nucleotide overhang of 1 to 4 nucleotides in length.

9. The pharmaceutical composition of claim 8, wherein the nucleotide overhang is one or two nucleotides in length.

10. The pharmaceutical composition of claim 8, wherein the nucleotide overhang is on the 3′-terminus of the first complementary RNA strand.

11. The pharmaceutical composition of claim 8, wherein the dsRNA further comprises a first end and a second end, wherein the first end comprises the 3′-terminus of the first complementary RNA strand and the 5′-terminus of the second RNA strand, and wherein the second end comprises the 5′-terminus of the first complementary RNA strand and the 3′-terminus of the second RNA strand, wherein the first end comprises a nucleotide overhang on the 3′-terminus of the first complementary RNA strand, and wherein the second end is blunt.

12. The pharmaceutical composition of claim 11, wherein the first complementary RNA strand is 23 nucleotides in length and comprises a 2-nucleotide overhang at the 3′-terminus, wherein the second RNA strand is 21 nucleotides in length, the wherein the second end of the dsRNA is blunt.

13. The pharmaceutical composition of claim 1, wherein the pharmaceutically acceptable carrier is an aqueous solution.

14. The pharmaceutical composition of claim 13, wherein the aqueous solution is phosphate buffered saline.

15. The pharmaceutical composition of claim 1, wherein the pharmaceutically acceptable carrier comprises a micellar structure selected from the group consisting of a liposome, capsid, capsoid, polymeric nanocapsule, and polymeric microcapsule.

16. The pharmaceutical composition of claim 15, wherein the polymeric nanocapsule and polymeric microcapsule comprise polybutylcyanoacrylate.

17. The pharmaceutical composition of claim 1, which is formulated to be administered by inhalation, infusion, injection, or orally.

18. The pharmaceutical composition of claim 1, which is formulated to be administered by intravenous or intraperitoneal injection.

19. A method for inhibiting the expression of a target gene in a mammal, which comprises administering a pharmaceutical composition comprising a double-stranded ribonucleic acid (dsRNA) and a pharmaceutically acceptable carrier, wherein the dsRNA comprises a nucleotide sequence which is substantially identical to at least a part of the target gene and which is less than 25 nucleotides in length, and wherein the pharmaceutical composition is in a unit dosage amount of less than 5 milligram (mg) of dsRNA per kg body weight of the mammal.

20. The method of claim 19, wherein the dosage unit of dsRNA is in a range of 0.01 to 2.5 milligrams, 0.1 to 200 micrograms, 0.1 to 100 micrograms, 1.0 to 50 micrograms, or 1.0 to 25 micrograms per kilogram body weight.

21. The method of claim 20, wherein the dosage unit of dsRNA is less than 25 g per kilogram body weight.

22. The method of claim 20, wherein the dsRNA further comprises a complementary RNA strand, wherein the complementary RNA strand comprises a complementary nucleotide sequence which is complementary to an mRNA transcript of a portion of the target gene, and wherein the complementary nucleotide sequence is 19 to 24 nucleotides in length, 20 to 24 nucleotides in length, or 21 to 23 nucleotides in length.

23. The method of claim 22, wherein the complementary nucleotide sequence is 22 or 23 nucleotides in length.

24. The method of claim 22, wherein the complementary RNA strand is 21 to 30 nucleotides in length, 21 to 25 nucleotides in length, or 21 to 24 nucleotides in length.

25. The method of claim 22, wherein the complementary RNA strand is 23 nucleotides in length.

26. The method of claim 19, wherein the dsRNA comprises a first complementary RNA strand and a second RNA strand, wherein the first complementary RNA strand comprises a complementary nucleotide sequence which is complementary to an RNA transcript of a portion of the target gene, and wherein the first complementary RNA strand and the second RNA strand comprise a 3′-terminus and a 5′-terminus, and wherein at least one of the first RNA and second RNA strands comprise a nucleotide overhang of 1 to 4 nucleotides in length.

27. The method of claim 26, wherein the nucleotide overhang is one or two nucleotides in length.

28. The method of claim 26, wherein the nucleotide overhang is on the 3′-terminus of the first complementary RNA strand.

29. The method of claim 26, wherein the dsRNA further comprises a first end and a second end, wherein the first end comprises the 3′-terminus of the first complementary RNA strand and the 5′-terminus of the second RNA strand, and wherein the second end comprises the 5′-terminus of the first complementary RNA strand and the 3′-terminus of the second RNA strand, wherein the first end comprises a nucleotide overhang on the 3′-terminus of the first complementary RNA strand, and wherein the second end is blunt.

30. The method of claim 29, wherein the first complementary RNA strand is 23 nucleotides in length and comprises a 2-nucleotide overhang at the 3′-terminus, wherein the second RNA strand is 21 nucleotides in length, the wherein the second end of the dsRNA is blunt.

31. The method of claim 29, wherein the pharmaceutically acceptable carrier is an aqueous solution.

32. The method of claim 31, wherein the aqueous solution is phosphate buffered saline.

33. The method of claim 29, wherein the pharmaceutically acceptable carrier comprises a micellar structure selected from the group consisting of a liposome, capsid, capsoid, polymeric nanocapsule, and polymeric microcapsule.

34. The method of claim 33, wherein the polymeric nanocapsule and polymeric microcapsule comprise polybutylcyanoacrylate.

35. The method of claim 29, which is formulated to be administered by inhalation, infusion, injection, or orally.

36. The method of claim 29, which is formulated to be administered by intravenous or intraperitoneal injection.

37. A method for treating a disease caused by the expression of a target gene in a mammal, which comprises administering a pharmaceutical composition comprising a double-stranded ribonucleic acid (dsRNA) and a pharmaceutically acceptable carrier, wherein the dsRNA comprises a nucleotide sequence which is substantially identical to at least a part of the target gene and which is less than 25 nucleotides in length, and wherein the pharmaceutical composition is in a unit dosage amount of less than 5 milligram (mg) of dsRNA per kg body weight of the mammal.

38. The method of claim 37, wherein the unit dosage amount of dsRNA is in a range of 0.01 to 2.5 milligrams, 0.1 to 200 micrograms, 0.1 to 100 micrograms, 1.0 to 50 micrograms, or 1.0 to 25 micrograms per kilogram body weight.

39. The method of claim 37, wherein the unit dosage amount of dsRNA is less than 25 micrograms per kilogram body weight.

40. The method of claim 37, wherein the dsRNA further comprises a complementary RNA strand, wherein the complementary RNA strand comprises a complementary nucleotide sequence which is complementary to an mRNA transcript of a portion of the target gene, and wherein the complementary nucleotide sequence is 19 to 24 nucleotides in length, 20 to 24 nucleotides in length, or 21 to 23 nucleotides in length.

41. The method of claim 40, wherein the complementary nucleotide sequence is 22 or 23 nucleotides in length.

42. The method of claim 40, wherein the complementary RNA strand is 21 to 30 nucleotides in length, 21 to 25 nucleotides in length, or 21 to 24 nucleotides in length.

43. The method of claim 40, wherein the complementary RNA strand is 23 nucleotides in length.

44. The method of claim 37, wherein the dsRNA comprises a first complementary RNA strand and a second RNA strand, wherein the first complementary RNA strand comprises a complementary nucleotide sequence which is complementary to an RNA transcript of a portion of the target gene, and wherein the first complementary RNA strand and the second RNA strand comprise a 3′-terminus and a 5′-terminus, and wherein at least one of the first RNA and second RNA strands comprise a nucleotide overhang of 1 to 4 nucleotides in length.

45. The method of claim 44, wherein the nucleotide overhang is one or two nucleotides in length.

46. The method of claim 44, wherein the nucleotide overhang is on the 3′-terminus of the first complementary RNA strand.

47. The method of claim 44, wherein the dsRNA further comprises a first end and a second end, wherein the first end comprises the 3′-terminus of the first complementary RNA strand and the 5′-terminus of the second RNA strand, and wherein the second end comprises the 5′-terminus of the first complementary RNA strand and the 3′-terminus of the second RNA strand, wherein the first end comprises a nucleotide overhang on the 3′-terminus of the first complementary RNA strand, and wherein the second end is blunt.

48. The method of claim 47, wherein the first complementary RNA strand is 23 nucleotides in length and comprises a 2-nucleotide overhang at the 3′-terminus, wherein the second RNA strand is 21 nucleotides in length, the wherein the second end of the dsRNA is blunt.

49. The method of claim 37, wherein the pharmaceutically acceptable carrier is an aqueous solution.

50. The method of claim 49, wherein the aqueous solution is phosphate buffered saline.

51. The method of claim 37, wherein the pharmaceutically acceptable carrier comprises a micellar structure selected from the group consisting of a liposome, capsid, capsoid, polymeric nanocapsule, and polymeric microcapsule.

52. The method of claim 51, wherein the polymeric nanocapsule and polymeric microcapsule comprise polybutylcyanoacrylate.

53. The method of claim 37, which is formulated to be administered by inhalation, infusion, injection, or orally.

54. The method of claim 37, which is formulated to be administered by intravenous or intraperitoneal injection.
